(19) United States
(12) Patent Application Publication
(10) Pub. No.: US 2006/0281076 A1
(43) Pub. Date: Dec. 14, 2006

(54) Substrate functionalization method for high sensitivity applications

(75) Inventors: Sudhakar S. Marla, Evanston, IL (US); Yijia Paul Bao, Vernon Hills, IL (US); Martin Huber, Evanston, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Nanosphere, Inc.

(21) Appl. No.: 11/436,395

(22) Filed: May 18, 2006

Related U.S. Application Data
(60) Provisional application No. 60/682,159, filed on May 18, 2005.

(51) Int. Cl. C12Q 1/70; C12Q 1/68; G01N 33/53; C12M 1/34; B05D 3/02

(52) U.S. Cl. 435/5; 435/6; 435/7.1; 435/287.2; 427/2.11; 977/924

(57) ABSTRACT

A method for functionalizing substrates such as magnetic beads and glass slides is provided. The method involves providing substrate surfaces with activated polyacrylic acid (PAA) and attaching one or more desired capture probes. Substrates prepared by the method are particularly useful in ultrasensitive detection assays as they exhibit very low specific binding and high binding efficiency relative to conventional substrates.

### CROSS-REFERENCE

This application claims the benefit of priority to U.S. provisional application Ser. No. 60/682,159, filed May 18, 2005, which is incorporated by reference in its entirety.

### FIELD OF THE INVENTION

The present invention relates to substrates and method for functionalizing substrates such as magnetic beads and glass slides to allow for efficient conjugation of bio-molecules such as DNA and proteins. The functionalized beads offer unique advantages including very low non-specific binding and high binding efficiency and are optimal for use in ultra-high sensitivity applications such as the bio-barcode assay.

### BACKGROUND OF THE INVENTION

Substrate modification plays an important role in biomolecule detection technology for controlling background as well as spot morphology in the case of array substrates. Paramagnetic beads in the micron to sub-micron ranges have been used as substrates in a number of biological applications including bio-molecule isolation and in bio-molecule detection assays. In detection assays, typically, antibodies or DNA oligonucleotides complementary to targets attached to the surface of magnetic beads are used to ‘fish out’ analytes from solution. Because of their size they can be retained in suspension for long periods and therefore participate in pseudo-solution reactions where they can capture low-target analytes of interest. Once the analyte is bound, the magnetic beads can be isolated from the solution by using a magnetic field.

Typically, the magnetic beads have an overcoat of silica or polymeric material which may have functional groups for attachment. Attachment of antibodies or DNA to the surface of the particle can be of a non-specific nature or it may involve chemical coupling to form covalent bonds. Covalent bonds are formed typically by activating the functional groups on the bead surface (for example to an NHS ester or tosyl group) and reacting with an amine-functionalized oligonucleotide or an antibody. The performance of the modified magnetic beads in assays may be judged by their binding capacity of the target analyte as compared with their non-target binding capacity.

For most applications, the available coupling methods provide reasonable signal-to-noise values translating to satisfactory results. In high sensitivity applications such as the Bio-barcode assay, it is important that the beads are able to efficiently bind extremely low copies of the target, which may be on the order of 10-1000 copies. (see, “Nanoparticle-Based Detection in Cerebral Spinal Fluid of a Soluble Pathogenic Biomarker for Alzheimer's Disease.” Georganopoulou, D. G., Chang, L., Nam, J.-N., Thaxton, C. S., Mufson, E. J., Klein, W. L., Mirkin, C. A. Proc. Nat. Acad. Sciences (2005) 102, (7), 2263-2264; Nanoparticle-Based Bio-Bar Codes for the Ultrasensitive Detection of Proteins Nam J.-M., Thaxton, C. S., and Mirkin, C. A. Science (2003) 301, 1884-1886; Bio-Barcodes Based on Oligonucleotide-Modified Nanoparticles Nam, J.-M., Park, S.-J., and Mirkin, C. A. J. Am. Chem. Soc. (2002) 124(15); 3820-3821.) See also, U.S. Ser. No. 10/877,750, filed Jun. 25, 2004 and U.S. Ser. No. TBA, filed May 12, 2005 (Attorney Ref. No. 03-666-G), which are incorporated by reference in its entirety. More importantly, the associated non-specific binding needs to be vanishingly low because even a few amplifier probes bound non-specifically to the magnetic bead can negatively affect the outcome of the assay; the reporter barcodes from the non-specifically bound amplifier probes can result in false positives. In cases involving nanoparticle-labeled probes, particularly gold nanoparticle probes, for detection of target analytes on capture substrates, the detection of extremely low amounts of target analytes in a sample may be complicated by a relative high background signal due to non-specific binding of the nanoparticle-based detection probes onto substrate surfaces in a chip-based assay.

Accordingly, maximizing signal and minimizing noise remain the key determinants of successful detection assays. Their importance takes even higher importance in the design of ultra-sensitive assays where the analyte is in low abundance. A methodology to conjugate capture probes such as antibodies to substrates such as paramagnetic beads that enhances signals in the assay by improving binding efficiency and offers significant noise reduction by providing a surface with minimal non-specific binding would be highly desirable.

### SUMMARY OF THE INVENTION

The present invention relates to a coating methodology for substrates such as magnetic beads that not only improves binding efficiency but dramatically decreases non-specific binding in ultra-sensitive detection applications. The underlying theme involves covalently attaching a hydrophilic polymer coating to the surface of the magnetic bead or other substrate wherein the polymer contains multiple activated functional groups. The activated groups on the polymers are reacted with molecules such as antibodies or nucleic acids to generate functionalized magnetic beads and other substrates. Modified substrate surfaces of the invention have surprising reduced non-specific binding and background in detection assays, particularly ultra-sensitive detection assays.

In one embodiment of the invention, a method is provided for modifying a substrate surface, the surface comprising optional amino groups, said method comprising: (a) providing an activated form of polyacrylic acid; and (b) contacting a substrate surface with the activated form of polyacrylic acid to form an activated polyacrylic acid coated surface.

In one aspect, amino groups are present on the surface.

In another aspect, no amino groups are present on the surface.

In another embodiment of the invention, a method is provided for modifying a substrate surface, the surface comprising optional displaceable functional groups, said method comprising: (a) contacting the substrate surface with polyacrylic acid to form a polyacrylic acid-containing surface; and (b) activating the polyacrylic acid-containing surface to form an activated form of polyacrylic acid coating on said surface.

In one aspect, the displaceable functional group is present.

In another aspect, the displaceable functional group comprises a tosyl or mesyl group.

In another aspect, the surface does not include a displaceable functional group.

In any of the above methods, the activated form of polyacrylic acid comprises an N-hydroxysuccinimide ester of polyacrylic acid or N-hydroxysulfosuccinimide ester of polyacrylic acid.

In another embodiment of the invention, the method further comprises contacting at least a portion of the activated polyacrylic acid coated surface with a molecule so as to immobilize the molecule onto the surface.

In one aspect, the molecule comprises DNA, RNA, polypeptide, antibody, antigen, carbohydrate, protein, peptide, amino acid, carbohydrate, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, metabolites of or antibodies to any of the above substances. The nucleic acids and oligonucleotides comprise genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, natural and synthetic nucleic acids.

In another aspect, the molecule is a member of a specific binding pair comprising antigen and antibody-specific binding pairs, biotin and avidin binding pairs, carbohydrate and lectin bind pairs, complementary nucleotide sequences, complementary peptide sequences, effector and receptor molecules, enzyme cofactor and enzymes, and enzyme inhibitors and enzymes.

In another of the above methods, the substrate has a plurality of different molecules attached thereto in an array to allow for the detection of multiple types of target analytes. The substrate includes magnetic beads, glass slides, silica beads, microplate well, beads, polymer membrane, or optical fiber.

In another embodiment of the invention, a modified substrate is provided by any of the inventive methods described herein.

In one aspect, modified substrates include array substrates for use in the ultra-sensitive detection of target analytes such as nucleic acid molecules or proteins.

In another embodiment of the invention, an improved method is provided for detecting at least one target analyte in a sample, the improvement comprising contacting the sample with a substrate having a bound molecule comprising a binding complement specific to the target analyte, the substrate prepared by any of the above methods.

In another embodiment, a method is provided for detecting for at least one target analyte, the target analyte having at least two binding sites, in a sample, the method comprising steps of: (a) incubating a capture probe, the sample and a detection probe under conditions effective to allow complex formation between the capture probe, the target analyte, and the detection probe, wherein (i) the capture probe comprising a molecule bound to the magnetic bead, the molecule comprising a first binding complement specific to the target analyte, the magnetic bead having a surface modified by any one of the methods of claims 1 or 4, (ii) the detection probe comprises a gold nanoparticle, a second binding complement to the target analyte bound to the nanoparticle, and reporter moieties bound to the nanoparticle; (b) separating the complex from any unbound detection probe; (c) selectively releasing at least a portion of the reporter moieties from the complex; and (d) analyzing the presence or absence of the reporter moieties, wherein the presence or absence of reporter moieties is indicative of the presence or absence of the target analyte.

In one aspect, the complex is separated from any unbound detection probe by the application of a magnetic field.

In another aspect, the nanoparticles include oligonucleotides bound thereto, the reporter moieties comprise oligonucleotides complementary to at least a portion of the oligonucleotides, and the reporter moieties are selectively released from the complex by dehybridization.

In yet another aspect, the reporter moieties are directly or indirectly bound to the nanoparticles.

These and other embodiments of the invention will be apparent in light of the detailed description below.

### DESCRIPTION OF THE FIGURES

FIG. 1 illustrates the process of preparing a conjugated substrate.

FIG. 2 illustrates the process of making NHS activated poly(acrylic acid)

FIG. 3 illustrates high sensitivity detection of HBV target in a DNA biobarcode assay. Reliable detection of 450 copies of an HBV target was demonstrated using polyacrylic acid coated magnetic beads.

FIG. 4 illustrates high sensitivity detection of PSA in a protein biobarcode assay. Reliable detection of 2 picograms quantities above background is possible with the polyacrylic acid coated magnetic particles.

FIG. 5 illustrates a microarray based allele specific hybridization of human genomic DNA on PAA modified glass surface. Three single nucleotide polymorphisms (SNPs; located in the F5, F2 and Mthfr gene) were genotyped using the indicated amount of human genomic DNA. After hybridizing the target DNA the bound nucleic acid was detected by a second hybridization utilizing oligonucleotide modified gold nanoparticles. Following a signal enhancement step, the hybridization was visualized by capturing the scatter signal.

FIG. 6 illustrates the extremely low non-specific binding associated with PAA coated magnetic beads when compared to commercially available amine-modified and carboxylic acid-modified magnetic beads. Image shows Well 1, PAA (50,000 MW) coated beads. Well 2, PAA (2,000 MW) coated beads. Well 3, Amine-modified beads. Well 4, Carboxylic acid-modifed beads. Well 5, no barcode ctrl. The PAA coated magnetic beads (wells 1,2) appear similar to the no barcode ctrl (well 5).

### DESCRIPTION OF THE INVENTION

All patents, patent applications, and references cited herein are incorporated by reference in their entirety.

As defined herein, the term “polyacrylic acid” (PAA) refers to acrylic acid polymers having a formula [—CH₂CH(CO₂H)—]. These polymers are commercially available from a variety of sources (e.g., Aldrich Chemicals, St. Louis, Mo., USA) in average molecular weights generally ranging from 2000 to 4,000,000 in the form of powders and % solution in water either as free acid or as a sodium salt.

The term “activated form of polyacrylic acid” refers to polyacrylic acid that has been activated by reaction with chemical reagents to form multiple reactive groups or sites in the polymer such as reactive ester groups. These reactive sites are available for attaching the polymer to the substrate surface as well as conjugation to molecules.

The term “substrate” refers any solid support suitable for coating with PAA or the activated form of PAA and for immobilizing oligonucleotides and other molecules. These include nylon, nitrocelluose, activated agarose, diazotized cellulose, latex particles, plastic, polystyrene, glass and polymer coated surfaces. These solid supports are used in many formats such as slides, membranes, microtiter plates, beads, probes, dipsticks, optical fibers, etc. Of particular interest to the present invention is the use of magnetic beads which have been used in the biobarcode assay described in U.S. Ser. No. 11/127,808, filed May 12, 2005 and PCT/US05/16545, filed May 12, 2005 as well as the use of glass and nylon surfaces in the preparation of DNA microarrays which have been described in recent years (Ramsay, Nat. Biotechnol., 16: 40-4 (1998)). The journal Nature Genetics has published a special supplement describing the utility and limitations of microarrays (Nat.Genet., 21(1): 1-60 (1999). Also of interest are optical substrates such as the ones described in U.S. Pat. No. 6,807,352, which is incorporated by reference in its entirely. Preferably the use of any solid support requires the presence of a nucleophilic group to react with the activated form of polyacrylic acid which contain “reactive groups” capable of reacting with the nucleophilic group. Suitable nucleophilic groups or moieties include hydroxyl, sulfhydryl, and amino groups or any moiety that is capable of coupling with the polyacrylic acid of the invention. Chemical procedures to introduce the nucleophilic or the reactive groups onto solid support are known in the art, they include procedures to activate nylon (U.S. Pat. No. 5,514,785), glass (Rodgers et al., Anal. Biochem., 23-30 (1999)), agarose (Highsmith et al., J., Biotechniques 12: 418-23 (1992) and polystyrene (Gosh et al., Nuc. Acid Res., 15: 5353-5372 (1987)). The preferred substrate is glass.

The substrates may have surfaces that are porous or non-porous. As defined herein, the term “porous” means surface means that the surface permits diffusion to occur. The term “non-porous” surface means that the surface does not permit diffusion to occur.

The term “analyte,” or “target analyte”, as used herein, is the substance to be quantitated or detected in the test sample using substrates prepared by the method of the present invention. The analyte can be any substance for which there exists a naturally occurring specific binding member (e.g., an antibody, polypeptide, DNA, RNA, cell, virus, etc.) or for which a specific binding member can be prepared, and the analyte can bind to one or more specific binding members in an assay.

The term “molecule” refers to any desired substance, such as a desired specific binding member, that may be immobilized onto the surface of the substrate. The “specific binding member,” as defined herein, means either member of a cognate binding pair. A “cognate binding pair,” as defined herein, is any ligand-receptor combination that will specifically bind to one another, generally through non-covalent interactions such as ionic attractions, hydrogen bonding, Vanderwaals forces, hydrophobic interactions and the like. Exemplary cognate pairs and interactions are well known in the art and include, by way of example and not limitation: immunological interactions between an antibody or Fab fragment and its antigen, hapten or epitope; biochemical interactions between a protein (e.g. hormone or enzyme) and its receptor (for example, avidin or streptavidin and biotin), or between a carbohydrate and a lectin; chemical interactions, such as between a metal and a chelating agent; and nucleic acid base pairing between complementary nucleic acid strands; a peptide nucleic acid analog which forms a cognate binding pair with nucleic acids or other PNAs. Thus, a molecule may be a specific binding member selected from the group consisting of antigen and antibody-specific binding pairs, biotin and avidin binding pairs, carbohydrate and lectin bind pairs, complementary nucleotide sequences, complementary peptide sequences, effector and receptor molecules, enzyme cofactor and enzymes, and enzyme inhibitors and enzymes. Other specific binding members include, without limitation, DNA, RNA, polypeptide, antibody, antigen, carbohydrate, protein, peptide, amino acid, carbohydrate, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, metabolites of or antibodies to any of the above substances. Nucleic acids and oligonucleotides comprise genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, natural and synthetic nucleic acids, and aptamers. Preparation of antibody and oligonucleotide specific binding members is well known in the art. Molecules may be immobilized onto substrates surfaces coated with activated form of polyacrylic acid and serve as capture probes for target analytes. Molecules may also include a detection label such as a fluorophore or nanoparticle. The molecules (M) have at least one or more nucleophilic groups, e.g., amino, carboxylate, or hydroxyl, that are capable of linking or reacting with the activated form of polyacrylic acid to so that they can be immobilized onto the surfaces of substrates. These nucleophilic groups are either already on the molecules or are introduced by known chemical procedures.

The term “capture probe” refers to any antibody, oligonucleotide, lectin or similar material that is capable of selectively and specifically binding to the target species of interest. Capture probe includes molecules as defined herein. Target analytes such as proteins, polypeptides, fragments, variants, and derivatives may be used to prepare antibodies using methods known in the art. Antibodies may be polyclonal, monospecific polyclonal, monoclonal, recombinant, chimeric, humanized, fully human, single chain and/or bispecific.

Polyclonal antibodies directed toward a target analyte generally are raised in animals (e.g., rabbits or mice) by multiple subcutaneous or intraperitoneal injections of JNK activating phosphatase polypeptide and an adjuvant. It may be useful to conjugate an target analyte protein, polypeptide, or a variant, fragment or derivative thereof to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for anti-target analyte antibody titer.

Monoclonal antibodies directed toward target analytes are produced using any method that provides for the production of antibody molecules by continuous cell lines in culture. Examples of suitable methods for preparing monoclonal antibodies include hybridoma methods of Kohler, et al., Nature 256:495-97 (1975), and the human B-cell hybridoma method, Kozbor, J. Immunol. 133:3001 (1984); Brodeur et al., Monoclonal Antibody Production Techniques and Applications 51-63 (Marcel Dekker 1987).

The term “oligonucleotide” referred to herein includes naturally occurring, and modified nucleotides linked together by naturally occurring, and/or non-naturally occurring oligonucleotide linkages. Oligonucleotides are a polynucleotide subset comprising members that are generally single-stranded and have a length of 200 bases or fewer. In certain embodiments, oligonucleotides are 10 to 60 bases in length. In certain embodiments, oligonucleotides are 10, 11, 12, 13, 14, 15, 16, 17, 18, 19, or 20 to 40 bases in length. Oligonucleotides may be single stranded or double stranded, e.g. for use in the construction of a gene mutant. Oligonucleotides of the invention may be sense or antisense oligonucleotides with reference to a protein-coding sequence.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “analyte” or “target analyte” refers to a substance to be detected or assayed by the method of the invention. Typical analytes may include, but are not limited to proteins, peptides, nucleic acid segments, molecules, cells, microorganisms and fragments and products thereof, or any substance for which attachment sites, binding members or receptors (such as antibodies) can be developed. The analytes have at least one binding site, preferably at least two binding sites, e.g., epitopes, that can be targeted by a capture probe and a detection probe, e.g. antibodies.

“Nanoparticles” useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs. The size of the nanoparticles is preferably from about 5 nm to about 150 nm (mean diameter), more preferably from about 5 to about 50 nm, most preferably from about 10 to about 30 nm. The nanoparticles may also be rods. Other nanoparticles usefuil in the invention include silica and polymer (e.g. latex) nanoparticles. Gold nanoparticles are preferred.

Methods of making metal, semiconductor and magnetic nanoparticles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Taransactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Methods of making silica nanoparticles impregnated with fluorophores or phosphors are also well known in the art (see Tan and coworkers, PNAS, 2004, 101, 15027 - 15032). Method for making gold nanoparticles are described, for instance, in U.S. Pat. Nos. 6,677,122 and 6,720,411 which are incorporated by reference in their entirety.

Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky et al., J. Am. Chem. Soc., 112, 9438 (1990); Ushida et al., J. Phys. Chem., 95, 5382 (1992).

As used herein, the terms “label” or “detection label” refers to a detectable marker that may be detected by photonic, electronic, opto-electronic, magnetic, gravity, acoustic, enzymatic, or other physical or chemical means. The term “labeled” refers to incorporation of such a detectable marker (e.g. by incorporation of a radiolabeled nucleotide or attachment to a reporter moiety, e.g, biobarcode).

A “sample” as used herein refers to any quantity of a substance that comprises potential target analytes and that can be used in a method of the invention. For example, the sample can be a biological sample or can be extracted from a biological sample derived from humans, animals, plants, fungi, yeast, bacteria, viruses, tissue cultures or viral cultures, or a combination of the above. They may contain or be extracted from solid tissues (e.g. bone marrow, lymph nodes, brain, skin), body fluids (e.g. serum, blood, urine, sputum, seminal or lymph fluids), skeletal tissues, or individual cells. Alternatively, the sample can comprise purified or partially purified nucleic acid molecules or proteins and, for example, buffers and/or reagents that are used to generate appropriate conditions for successfully performing a method of the invention.

The present invention provides a method for modifying substrates, substrates having surfaces modified by the inventive method, and methods for using the modified substrates. The method of the invention allows for efficient conjugation of molecules with reduced non-specific binding and enhanced binding efficiency. Substrates prepared by the inventive method are optimal for use in ultra-high sensitivity applications such as the biobarcode assay described in U.S. Ser. No. 11/127,808, filed May 12, 2005 and PCT/US05/16545, filed May 12, 2005. The use of the polyacrylic acid polymer provides a hydrophilic coating that minimizes non-specific binding including that observed due to gold nanoparticle probes and unmodified magnetic beads used in the biobarcode assay. The activated, e.g., N-hydroxysuccinimide (NHS), sites on the polymer are expected to be far more accessible to the antibody. In turn, the molecule, e.g., antibodies, bound to the magnetic beads should be more accessible to the target. Together, the improved accessibility coupled with increased number of antibodies on the magnetic beads increases the beads' target binding efficiency. Moreover, the stability of the bound antibody is also improved potentially because of the added flexibility associated with the polymer; the antibody is not bound to a solid surface but via a flexible polymer chain.

In one embodiment of the invention, a method is provided for modifying a substrate surface, the surface comprising optional amino groups, said method comprising: (a) providing an activated form of polyacrylic acid; and (b) contacting a substrate surface with the activated form of polyacrylic acid to form an activated polyacrylic acid coated surface. The modified activated surface may be used immediately for further modification as discussed below or stored for future use. As the activated substrate surface coating is sensitive to water, the stored coated substrate should be protected from water by any suitable means including an air-tight desiccator. Preferably the substrate surface includes an amino group for reacting with the activated form of polyacrylic acid.

In one aspect of the invention, the activated form of polyacrylic acid includes reactive esters such as esters of N-hydroxysuccinimide (NHS) or N-hydroxysulfosuccinimide (NHSS). Preferably a carbodiimide catalyst is used to catalyze formation of the active form where NHS or NHSS are used. Any suitable conditions and temperatures for modifying. PAA in order to activate it may be used. The preferred activated esters of polyacrylic acid include an N-hydroxysuccinimide ester of polyacrylic acid and an N-hydroxysulfosuccinimide ester of polyacrylic acid. These active forms of polyacrylic acid can be prepared by any suitable means. For instance, N-hydroxysuccinimide ester of polyacrylic acid may be prepared by reacting polyacrylic acid with N-hydroxysuccinimide [NHS] in the presence of a carbodiimide catalyst such as 1-ethyl-3-(3-dimethylaminopropyl)carbodiimide hydrochloride [EDC], dicyclohexyl carbodiimide (DCC), 1-cyclohexyl-3-(2-morpholinoethyl)carbodiimide [CMC], diusopropyl carbodiimide (DIC). In addition, the NHS in the above reaction can be replaced with the water-soluble N-hydroxysulfosuccinimide to yield the corresponding sulfo-NHS ester.

In another aspect of the invention PAA is activated form the O-acylisourea intermediate with 1-Ethyl-3-(3-dimethylaminopropyl)carbodiimide hydrochloride [EDC]; or 1-Cyclohexyl-3-(2-morpholinoethyl)carbodiimide [CMC]; or Diisopropyl carbodiimide (DIC). In another aspect of the invention PAA is activated by Carbonyldiimidazole (CDI) to form an N-Acylimidazol intermediate.

Different polymer lengths of PAA may be used for coating, the concentration of the available carboxylic acid groups is used to establish ratios for activation. The molar ratio of polyacrylic acid: N-hydroxysuccinimide or N-hydroxysulfosuccinimide: carbodiimide catalyst generally ranges from about 5 mM:_{—}100 mM:100 mM to about 500 mM:1 M:1 M, preferably about 250 mM:460 mM:460 mM. The reaction is generally carried out at temperatures ranging from 4° C. to 40° C., preferably about 25° C. Any suitable solvent may be used for preparing the activated form of polyacrylic acid. Representative examples of solvents include, without limitation, dimethylsulfoxide (DMSO) and dimethylformamide (DMF).

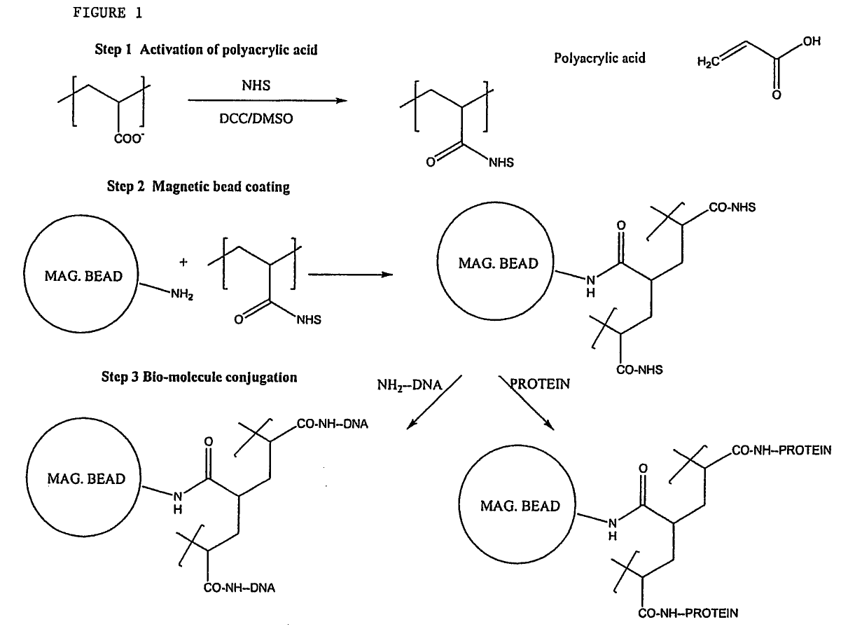

A representative activated form of polyacrylic acid is shown on FIG. 1. The carboxylic acid groups on the polyacrylic acid polymer chain are converted to activated esters such as activated NHS esters by a reaction with NHS catalyzed by DCC in DMSO (FIG. 1). A substrate such as magnetic beads contain a functional group such as amine functional groups on the surface which readily react with the activated polyacrylic acid polymer to generate a surface decorated with the activated polymer. Because of the stoichiometry of this reaction, the polymer retains most of the activated NHS groups which are available next for the conjugation reaction with molecules such as antibodies or oligonucleotides.

In another embodiment of the invention, a method is provided for modifying a substrate surface, the surface comprising optional displaceable functional groups, said method comprising: (a) contacting the substrate surface with polyacrylic acid to form a polyacrylic acid-containing surface; and (b) activating the polyacrylic acid-containing surface to form an activated form of polyacrylic acid coating on said surface. Any suitable optional displaceable function group, if present, may be used such as a tosyl group or mesyl group.

In one aspect, the substrate surface may be modified to include displaceable functional groups by any conventional method such directly modification or indirect modification such as the attaching linkers having a leaving or displaceable function group at the end not bound to the substrate. These displaceable functional groups may be displaced by the carboxylic acid functionality of the polyacrylic acid.

In another aspect, polyacrylic acid polymers can be attached to unmodified surfaces by methods including, but not limited to vapor deposition, coating by immersion, spin coating, spray coating, spotting, and painting. The attachment chemistry with these methods may be due to a multiplicity of interactions including but not limited to covalent interactions, non-covalent interactions, ionic interactions, adsorption, and absorption. The surfaces include but are not limited to metal, plastic, and glass. The PAA coating on the substrate is then converted into its activated form as described above.

For the conjugation reaction on surfaces of magnetic beads, the activated beads in solvent, e.g., DMSO, are isolated, washed in a suitable aqueous buffer and mixed with the aqueous solution of the molecule, e.g., antibody, of interest.

In another embodiment of the invention, a method is provided for modifying a substrate surface, the method comprising contacting at least a portion of the activated polyacrylic acid coated surface with a molecule so as to immobilize the molecule onto the surface. The molecule is dissolved in any suitable medium such as water, water-miscible organic solvent mixtures, buffers, and the like. The solution containing the molecules is then contacted onto at least a portion of the coated substrate by any suitable means, including without limitation, vapor deposition, coating by immersion, spin coating, spray coating, spotting, and painting. The modified substrate surface may include one or more different molecules. The molecule includes chemical functionality, e.g., amino groups, that would allow the molecule to react with the activated ester and form a bond to the substrate. For the conjugation reaction on surfaces of substrates such as magnetic beads, the activated beads in solvent, e.g., DMSO, are isolated, washed in a suitable aqueous buffer and mixed with aqueous solution of the molecule, e.g., antibody, of interest.

In one aspect, the modified substrate surface includes a plurality of different molecules attached thereto in an array to allow for the detection of multiple types of target analytes.

At the end of the attachment of the molecules onto the substrate, leftover activated carboxylic acid groups can be passivated by any suitable means including hydrolyzing the active group, affected by water, to expose the original carboxylic acid functional group. Alternately, molecules with free amino groups such as lysine or ethanolamine may be used to form an amide linkage and instead of a carboxylic acid end group provide a hydroxyl group. For instance, the passivation can be affected by incubation with 50 mM ethanolamine in appropriate buffers, for example Tris 0.1 M, pH 9 for 30 min at 25° C.

The above strategy has been utilized successfully in conjugating amine-modified DNA oligonucleotides onto magnetic beads. The first step, involving the generation of the activated magnetic bead surface is identical to that for the protein conjugation. Because the DNA is stable in organic solvents, the conjugation is conducted in DMSO. Once the conjugation is complete, the beads are isolated, washed repeatedly in water or aqueous buffer to remove unbound DNA.

In order to simplify the conjugation of proteins in aqueous media, the active NHS esters could be regenerated in situ. After the magnetic bead conjugation to the NHS-activated polymer, the beads would be washed to remove unbound polymer and brought into aqueous buffer. Any loss of active NHS groups due to hydrolysis would be compensated by coupling the protein in the presence of additional NHS (or the water-soluble equivalent NHSS and EDC. This strategy could also be used to couple amine-functionalized DNA oligonucleotides to the magnetic beads.

The overall approach is not restricted to magnetic beads. It may be used to functionalize all forms of microbeads and nanoparticles; the only requirement is that the beads or nanoparticles have available amine functional groups, which is easily accomplished by any number of methods available in the literature. The feasibility of this methodology has been demonstrated with silica beads with varied dimensions (150 nm, 350 nm, and 500 nm diameter). The density of the active NHS groups, and in turn the number of binding sites on the beads (and the different surfaces) can be modulated by increasing or decreasing the lengths of the polymer and changing the ratio of input NHS to the carboxylic acids. Extended further, the chemistry can be used to functionalize surfaces such as glass, plastic, metal, etc. where the purpose is to make available a hydrophilic surface that is able to bind molecules via an activated NHS-ester. The molecules that bind to the surface may be bio-molecules (DNA, proteins, carbohydrates, etc) or other chemical entities that recognize analytes such as “Molecularly Imprinted Polymers”. The chemistry may be used for purposes other than analyte detection. By modifying the surface chemistry the properties of the surface can be changed to increase or decrease hydrophilicity. This methodology has been extended to the preparation of array substrates for detecting target analytes.

The modified substrates of the invention are particularly useful in detection methods that are based on the use of gold nanoparticles probes for detecting at least one target analyte in a sample. These methods include contacting the sample with a substrate having a bound molecule comprising a binding complement specific to the target analyte and detecting the analyte using gold nanoparticle based detection probes.

Recently, the ultrasensitive bio-barcode detection assay has been reported as disclosed in U.S. Ser. No. 11/127,808, filed May 12, 2005 and PCT/U505/16545, filed May 12, 2005., which are incorporated by reference in their entirety. This is a nanoparticle-based approach to the detection of protein and DNA targets (Nam J M, Thaxton C S, Mirkin C A Nanoparticle-based bio-bar codes for the ultrasensitive detection of proteins, Science 301 (5641):1884-1886 Sep. 26, 2003; Nam J M, Stoeva S I, Mirkin C A Bio-bar-code-based DNA detection with PCR-like sensitivity, J. Am. Chem. Soc. 126 (19):5932-5933 May 19, 2004.) The bio-bar-code assay takes advantage of two target-seeking probes. First, a magnetic probe, surface-functionalized with the appropriate molecule as a recognition element (e.g., monoclonal Ab for proteins and a complementary DNA oligomer for nucleic acid targets) captures the target analyte present in a small detection volume where the recognition elements far outnumber the target analyte. The magnetic particles make washing to remove unbound and non-specifically bound portions in the mixture simple and highly efficient. Next, gold nanoparticles with the appropriate surface-bound recognition element (poly- or monoclonal antibodies for proteins and a non-overlapping complementary DNA oligomer for nucleic acid targets) are added to the magnetic-target analyte hybrid structures. Recognition of the hybrid structures by the gold nanoparticles results in the formation of a “sandwich” structure. Importantly, in addition to the target analyte recognition elements, the gold nanoparticles also carry with them surface-bound DNA oligomers that are hybridized to their anti-parallel complements by DNA base pairing. The complement sequence, referred to as the “bio-barcode,” has a sequence that has been chosen to serve as a surrogate for the target of detection. As each gold nanoparticle carries with it hundreds to thousands of bio-bar-code strands, there is a huge amplification of the detection signal for each sandwiched target. The bio-bar-code is easily released from the nanoparticle surface in the last step of the assay and further amplified and detected using conventional DNA detection techniques. These biobar codes may be labeled as desired and serve as reporter moieties.

The bio-bar-code approach is impressive in terms of detection sensitivity with regard to detecting protein targets (aM sensitivities versus the typical pM sensitivities of ELISA). Further, the use of bio-bar-code assays has been demonstrated to be as sensitive for DNA targets as PCR, without the need for enzymatic amplification of the target sequence. The assay allows one to identify protein markers down to the low attomolar (about 20 copies in a 10 ul sample) concentration limit. Because non-specific nanoparticle binding to the magnetic bead probes may affect the level of signal/noise or signal/background, one significant advance would be to modify the surface of the magnetic beads to reduce non-specific binding to the magnetic bead, thus improving the sensitivity of the biobarcode assay. The subsequently released biobarcodes can then be detected by nanoparticle-based and conventional, e.g., fluorophore, detection methods.

In another embodiment of the invention, a method is provided for detecting for at least one target analyte, the target analyte having at least two binding sites, in a sample, the method comprising steps of: (a) incubating a capture probe, the sample and a detection probe under conditions effective to allow complex formation between the capture probe, the target analyte, and the detection probe, wherein (i) the capture probe comprising a molecule bound to the magnetic bead, the molecule comprising a first binding complement specific to the target analyte, the magnetic bead having a surface modified by any one of the methods of claims 1 or 4, (ii) the detection probe comprises a gold nanoparticle, a second binding complement to the target analyte bound to the nanoparticle, and reporter moieties bound to the nanoparticle; (b) separating the complex from any unbound detection probe; (c) selectively releasing at least a portion of the reporter moieties from the complex; and (d) analyzing the presence or absence of the reporter moieties, wherein the presence or absence of reporter moieties is indicative of the presence or absence of the target analyte. The complex is separated from any unbound detection probe by the application of a magnetic field.

In one aspect of the invention, the nanoparticles include oligonucleotides bound thereto, the reporter moieties comprise oligonucleotides complementary to at least a portion of the oligonucleotides, and the reporter moieties are selectively released from the complex by dehybridization. In another aspect, the reporter moieties are directly or indirectly bound to the nanoparticles.

### EXAMPLES

The following examples are representative of the invention and do not serve to limit the scope of the invention.

List of Abbreviations:

DCC—Dicyclohexylcarbodimide
NHS—N-Hydroxy succinimide
EDC—1-Ethyl-3-(3-dimeethylaminopropyl)-carbodiimide
DMSO—Dimethylsulfoxide

### Example 1

### Preparation of Functionalized Magnetic Beads

In this Example, a procedure is provided for preparing functionalized magnetic beads.

(a) Activation of Polyacrylic Acid

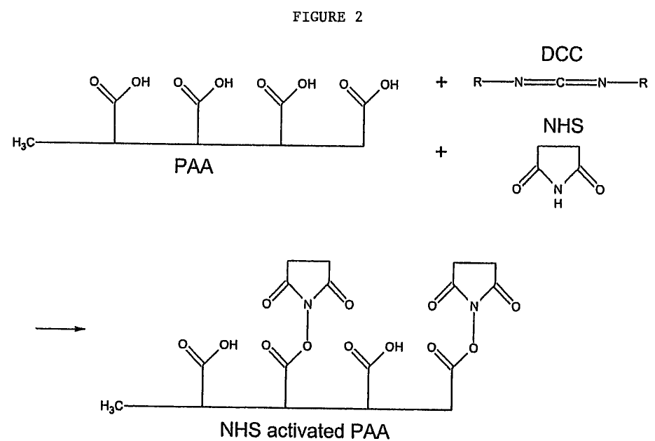

Polyacrylic acid (Aldrich, MW ˜2000) was dissolved in an 80:20 DMSO:Water mixture to give a 22% w/v solution. To 100 uL of this solution was added in succession, 600 uL of 1M solution of NHS prepared in DMSO and 600 uL of 1M solution of DCC prepared in DMSO. The solution was left overnight at RT with mild shaking. The resulting mixture containing the activated polymer and the DCC-urea precipitate was spun at 15,000 rcf for 10 min and the supernatant removed to a fresh tube. The step was repeated in order to ensure that the solution was clear of any particulate DCC-urea. Filtration to remove the DCC-urea is also an option for larger scale preparations. FIG. 2 illustrates the reaction.

(b) Coupling Activated Polymer to Amine-functionalized Magnetic Beads

Amine-functionalized magnetic beads (Polysciences) were re-suspended by gentle vortexing and 100 uL, the equivalent of 5 mg, was removed into a tube and washed 3× with 1000 uL water with the aid of a magnetic isolator rack. The water was replaced with 1000 uL DMSO, and the particles were washed next with DMSO 3× before being re-suspended in 1000 uL DMSO to yield a suspensions at 5 mg/mL. An equal amount of the activated polyacrylic acid was added to the tube and the mixture was incubated at RT for ˜6 h. The supernatant was removed after isolating the particles by using a magnetic isolator and the particles were washed repeatedly with DMSO (3×) before re-suspending the activated beads in DMSO at a final concentration of 5 mg/mL.

### Example 2

### Conjugation of Bio-molecules to the Magnetic Particles

In this Example, a generalized approach for the conjugation of bio-molecules to the magnetic particles is provided. See FIG. 1.

(a) DNA Coupling to the Activated Magnetic Beads

To 500 uL magnetic beads (5 mg/mL) was added 20 uL of a 0.2 mM amine-modified oligonucleotide solution and mixed vigorously on a vortex. The suspension was left overnight at RT with shaking. Finally, the beads were isolated, washed repeatedly with a wash buffer containing 50% formamide/0.01% Tween 20 at 50° C. repeatedly and re-suspend in 500 uL 2×SSC/15% Formamide/0.01% Tween to yield magnetic beads at an approximate concentration of 5 ug/uL.

(b) Protein Coupling to the Activated Magnetic Beads

To 2.5 mg of magnetic beads was added 323 uL of 1× PBS, 167 uL of 3 M ammonium sulfate, and 10 uL of antibody (10 ug/uL). The solution was mixed gently on a vortex and the suspension was incubated overnight at 37° C. on a rotisserie set at 30 rpm. After an overnight incubation, 50 uL of 10% BSA was added to the suspension and incubated for an additional 2 h at 37° C. with rotation (30 rpm). Finally, the beads were isolated, washed repeatedly with a wash buffer containing 1× PBS/0.05% Tween 20 at RT repeatedly and re-suspended in 500 uL 1× PBS/0.5% BSA/0.05% Tween 20 to yield magnetic beads at an approximate concentration of 5 ug/uL.

It has been observed that the presence of ammonium sulfate in the protein solution improves conjugation. Without limiting the invention by any theory of operation, it is believed that the ammonium sulfate condenses proteins onto the bead surface and increases the local concentration. It has also been noted that the ratio of the protein and the absolute concentration of the magnetic beads in the conjugation reaction should be monitored as very high protein concentrations and magnetic bead concentrations may lead to irreversible magnetic bead aggregation. Typical ranges of protein concentrations are 0.04 ug/uL and those for the magnetic beads are 0.5 ug/uL to 10 ug/uL.

In addition, completely anhydrous reaction conditions during the activation of polyacrylic acid by NHS in the presence of DCC gave poor results. The beads generated by using the activated polymer aggregated when brought into water from DMSO. Moreover, they were not functional in assays. To ensure that the NHS ester groups formed do not non-productively hydrolyze, the amount of water in the reaction is minimized. According to various accounts, the half-life of NHS is on the order of 1-2 h in water. However, the first step activation of the polyacrylic acid tolerated up to 7.5% v/v water in the DMSO background. It was found that the elimination of water completely resulted in beads aggregation, suggesting inadequate reaction conditions.

### Example 3

### DNA Detection Using the Functionalized Magnetic Particles

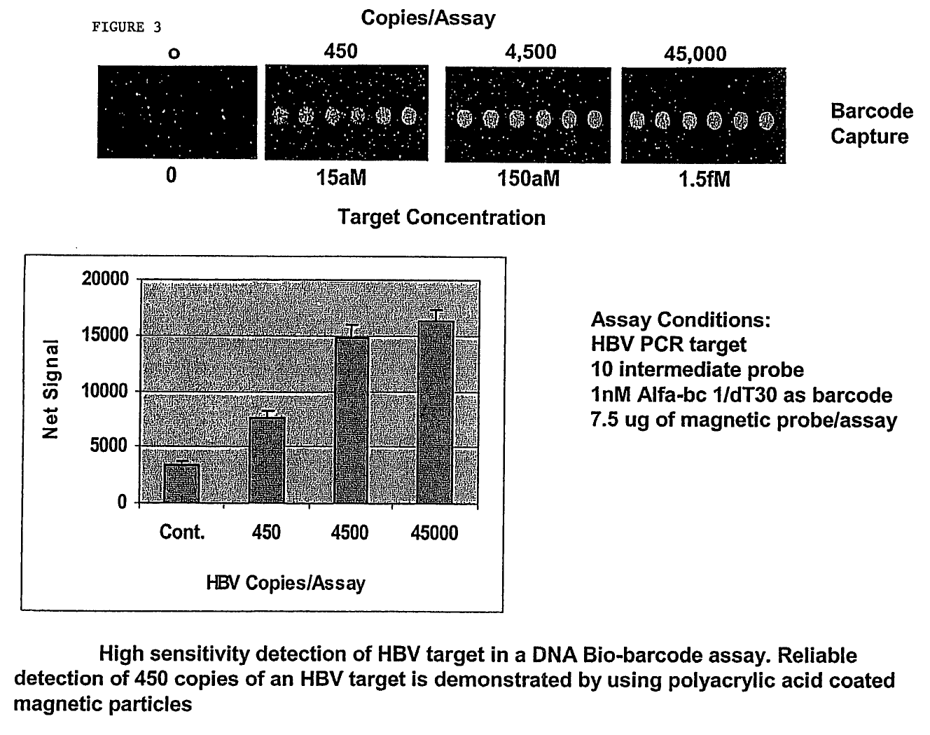

In this Example, a nucleic acid bio-barcode assay detecting low target concentrations HBV target is described. See FIG. 3. Capture oligonucleotides specific for the HBV target were conjugated to polyacrylic acid-coated paramagnetic particles. The low non-specific binding associated with the magnetic particles and their ability to capture low target amounts allows ultra-high sensitivity at ˜15 attomolar concentration (450 copies/assay).

Polymer-coated magnetic particles were functionalized with capture oligonucleotides specific to the HBV target. The HBV target was generated by PCR-amplifying the RNA target after a reverse transcription step. The magnetic beads (7.5 ug/assay) were added to a hybridization mix (final volume 50 uL) containing different concentrations of the HBV target 3×SSC/0.025% Tween 20/0.0125% SDS, 30% formamide, and 2 nM each of 17 intermediate oligos that had been previously denatured and incubated for 30 min at 40° C. The intermediate oligonucleotides are chimeras designed contained a part that is complementary to different regions of the PCR target and another part that is complementary to barcodes attached to a nanoparticle probe. The mixture containing the magnetic beads and target was incubated at 40° C. for 1 h with shaking. The beads were washed repeatedly (5×) by using a magnetic isolator and a wash solution (2×SSC/0.025% Tween 20/0.0125% SDS, 15% formamide). After the wash, to each tube was added the barcode-containing nanoparticle probe (100 pM final concentration in 3×SSC/0.025% Tween 20/0.0125% SDS, 30% formamide). The mixture was incubated for at 40° C. for 1 h with shaking and washed subsequently as described above and resuspended in water (50 uL). The barcodes from the barcode nanoparticle probe were released by the addition of DTT (1 mM final in water) and detected in an array-based hybridization assay. The array contained capture oligonucleotides complementary to one part of the barcode. Thus, the barcodes were hybridized to the DNA array in a hybridization mixture containing 3×SSC, 0.02% Tween 20, 0.0125% SDS, 30% formamide. To complete the assay, dT20mer coated gold-nanoparticles were added to bind the second part of the barcode—a dA region to form a sandwich and the hybridized array was stained with silver development solutions (Nanosphere, Inc., Northbrook, Ill.) and imaged with a light scattering based imaging system (e.g, Verigene ID®, Nanosphere Inc.). Detection sensitivity for the HBV detection are shown in FIG. 3.

### Example 4

### Protein Detection Using the Functionalized Magnetic Particles

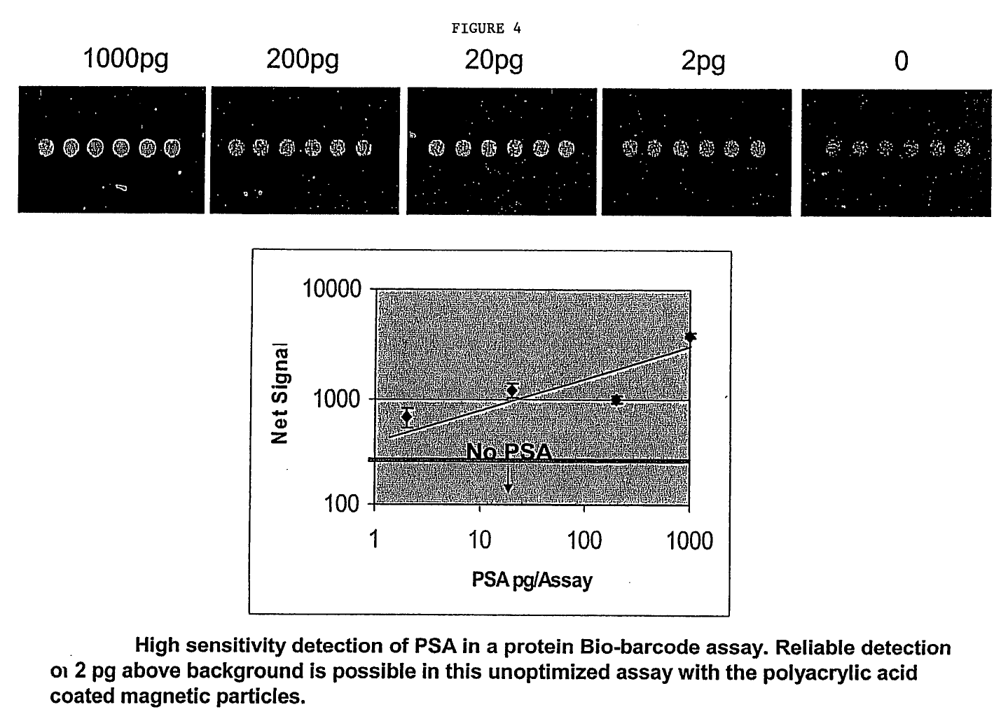

In this Example, a protein bio-barcode assay designed to detect low target concentrations of the Prostate Specific Antigen (PSA) is described. See FIG. 4. The polyacrylic acid coated magnetic particles were conjugated to a PSA monoclonal antibody via the NHS-activated carboxylic acid groups. High sensitivity detection of PSA (2 pg) is possible because of the low non-specific binding associated with the magnetic particles and their ability to capture low target amounts.

To capture PSA target, 10 μg of PSA antibody (BioDesign, Mab, α-PSA free formn) coated magnetic particle was incubated with the recombinant human Kallikrein 3 (rhPSA, R&D System) in a 50 μL volume of binding mixture containing 1× PBS, 0.5% BSA, 0.05% Tween 20, 6.6 μg/μL tRNA (Sigma) for 0.5-2 h at 25° C./1200 rpm. Then 100 ng of the biotinylated anti-human Kallikrein 3 polyclonal goat IgG, (anti-PSA-biotin Ab, R&D System) was added as secondary antibody and incubated for 0.5-2 h at 25° C./1200 rpm. The magnetic beads were washed with a wash buffer containing 1× PBS PBS, 0.04% Tween, 0.02% SDS, 0.05% BSA and resuspended in the binding mixture. To this suspension was added, 5 μL of the streptavidin coated nanoparticles (e.g, streptavidin coated 15 nm diameter gold particles, from BBI) and the. binding mixture and incubated for 0.5 h at 25° C./1200 rpm. After a wash step with the above-mentioned wash buffer to remove un-bound streptavidin coated nanoparticles, biotin-labeled barcodes, biotin-biotin-(dAdC)₁₅-dA ₂₅-biotin-biotin, were added to the binding mixture to load the streptavidin-coated nanoparticle probes. After a final wash step, the bound barcodes were released from streptavidin by heating in 95% formamide for 5 min at 65° C. (alternatively, the bound barcodes can be released in 95% formamide for 2 min at 90° C., or in 0.1% SDS for 5 min at 100° C.). The eluted barcodes were used for array hybridization. The barcodes were hybridized to a DNA array containing the probe sequence, (dGdT)₁₅, in a hybridization mixture containing 3×SSC, 0.02% Tween 20, 0.0125% SDS, 30% formamide. The dT20mer coated gold-nanoparticles are used to hybridize the dA region of the barcode sequence forming the “sandwich”. Finally, the hybridized array is stained with silver development solutions(Nanosphere, Inc., Northbrook, Ill.) and imaged with a light scattering based imaging system (e.g, Verigene ID®, Nanosphere Inc.). The intensities for the different concentrations of target are plotted in FIG. 4 showing the high sensitivity detection of PSA.

### Example 5

### Preparation of DNA Arrayed Plates

In this Example, a DNA arrayed plate is prepared using NHS activated PAA to modify an amine coated glass surface. DNA microarrays are widely used tools in modern molecular biology. The way the capture probes are immobilized onto the microarray surface greatly influences the hybridization reaction. Ideally the attachment chemistry provides a high loading capacity compared with low unspecific binding of reaction components. The described invention overcomes low hybridization efficiency by introducing a polymeric coating of a solid surface. Binding the capture probes to a polymer backbone significantly increase the hybridization rate and thereby improves the detection sensitivity. Additionally, the polymer coating of the surface exhibits very low unspecific binding of reaction compounds due to its highly hydrophilic nature.

The NHS activated PAA (see FIG. 2) can be used to coat any primary amine containing surface like amine modified glass surfaces, amine modified plastics or amine modified magnetic particles. Additionally the activated polymer can be attached non-covalently to other materials such as metal surfaces (e.g. gold surfaces). The resulting polymer coating provides a hydrophilic coating with a high loading capacity for biomolecules. The covalent attachment of such biomolecules occurs via the amine reactive NHS moiety of the activated polymer. The accessibility of the bound biomolecules to reaction partners is greatly increased due to the 3D like structure created by the polymeric backbone of the coating. Additionally, the hydrophilic character of the poly(acrylic acid) coating decreases unspecific binding of reaction partners thereby lowering the background signal. These facts increase the sensitivity of any assay significantly.

(a) Preparation of Functionalized Glass Substrate

A 0.05%-1% solution of poly(acrylic acid) (MW 10.000-50.000) is gently agitated in 500 mM N,N-Dicyclohexylcarbodimide (DCC) and 500 mM N-hydroxysuccinimide (NHS) in 95% DMSO for 12-24 hours at ambient temperature. After filtering the insoluble byproduct dicyclohexylurea the activated polymer is applied to an amine modified substrate. Coupling of the polymer to the substrate via the NHS ester is performed for 3-12 hours at ambient temperature. The surface is then washed in acetone, Ethanol followed by a brief wash in 0.01% SDS and a short rinse with de-ionized water. The polymer modified substrate is stored dry under Argon.

(b) Preparation of DNA Arrayed Plate

A 0.05%-1% solution of poly(acrylic acid) (MW 10.000-50.000) is gently agitated in 500 mM N,N-Dicyclohexylcarbodimide (DCC) and 500 mM N-hydroxysuccinimide (NHS) in 95% DMSO for 12-24 hours at ambient temperature. After filtering the insoluble byproduct dicyclohexylurea the activated polymer is applied to an amine modified substrate. Coupling of the polymer to the substrate via the NHS ester is performed for 3-12 hours at ambient temperature. The surface is then washed in Acetone, Ethanol followed by a brief wash in 0.01% aqueous SDS solution and a short rinse with de-ionized water. The polymer-modified substrate is stored dry under Argon. In order to attach DNA substrates on to the substrate, the desired amine-modified DNA capture oligonucleotides are dissolved in 150 mM Na-Phosphate buffer (pH 8.5) supplemented with 0.01% SDS at a concentration of anywhere between 10-500 mM concentration. The oligonucleotide mix is then deposited in an array format by using an arraying machine such as the Omnigrid (GeneMachines) arraying machine. The arrayed plate is then humidified overnight at 70% humidity and washed with 0.2% SDS followed by deionized water.

### Example 6

### PCR-less SNP Detection Using DNA Arrayed Plate

The resulting arrayed substrate prepared by using the protocol in Example 5 was then employed in the production of an oligonucleotide microarray used in multiplex SNP detection directly from human genomic DNA via allele specific hybridization. The SNPs interrogated in this example are Factor 2 (G20210A), Factor 5 (G1691A) and Mthfr (C677T). The sequences of the employed arrayed oligonucleotides are as follows:

| | | |
|---|---|---|
| 5′ CTCAGCGAGCCTCAATGC | (Factor II wt) | [SEQ ID NO.:1] |
| TCCC 3′ | | |
| 5′ CTCTCAGCAAGCCTCAAT | (Factor II mut) | [SEQ ID NO.:2] |
| GCTCC 3′ | | |
| 5′ GATGAAATCGGCTCCCGC | (Mthfr wt) | [SEQ ID NO.:3] |
| AGAC 3′ | | |
| 5′ ATGAAATCGACTCCCGCA | (Mthfr mut) | [SEQ ID NO.:4] |
| GACA 3′ | | |
| 5′ TGGACAGGCGAGGAATAC | (Factor V wt) | [SEQ ID NO.:5] |
| AGGTAT 3′ | | |
| 5′ CTGGACAGGCAAGGAATA | (Factor V mut) | [SEQ ID NO.:6] |
| CAGGTATT 3′ | | |

The sequences of the oligonucleotides tethered to the gold nanoparticles are as follows:

| | | |
|---|---|---|
| 5′ CCA CAG AAA ATG ATG | (Factor V) | [SEQ ID NO.:7] |
| CCC AGT GCT TAA CAA GAC | | |
| CAT ACT ACA GTG A 3′ | | |
| 5′ TCC TGG AAC CAA TCC | (Factor II) | [SEQ ID NO.:8] |
| CGT GAA AGA ATT ATT TTT | | |
| GTG TTT CTA AAA CT 3′ | | |
| 5′ GGA AGA ATG TGT CAG | (Mthfr) | [SEQ ID NO.:9] |
| CCT CAA AGA AAA GC 3′ | | |

The achieved sensitivity was sufficient to generate reliable hybridization signals from as little as 0.25 ug/ul of un-amplified human genomic DNA.

Human placental DNA (Sigma) or patient genomic DNA samples (Coriell Institute) were independently genotyped by sequencing methods. The DNA sample was fragmented by ultrasonication (Misonix), and conditions were adjusted to yield a median DNA length of ˜0.5 kb. The target hybridization mixture (5 μL) contained 4×SSC, 0.05% Tween 20, 35% formamide, and 0.5-5 μg human genomic DNA, or as indicated in the specific experiment. The hybridization mixture was added to the test well after a 3 min, 98° C. heat denaturation step. Each test slide possessed several sub-arrays that could be isolated by gaskets allowing for the testing of several test samples simultaneously. The test slide was incubated at 40° C. for 60 min and washed subsequently at room temperature twice (2 min each) in a wash buffer containing 0.5 M NaNO₃, 0.05% Tween 20. This low stringency wash was followed by a brief high stringency wash (30 s) in a low-salt wash buffer (0.4×SSC). Each sub-array was then covered with 50 uL hybridization buffer (4×SSC, 0.05% Tween 20, 35% formamide) containing gold-nanoparticle probes (1 nM ea) for 30 min at 40° C. The isolating gasket was removed and the test slide was washed again in the wash buffer for 3 min (2×) at RT with gentle agitation. Finally, the washed slide was stained with 2 mL of silver reagent, an admix of Silver enhancer A and B solutions (Sigma) for ˜5 min, washed in ddH₂O, and dried. The dried slide was imaged with a Nanosphere Verigene ID® (Nanosphere) imaging system or with an ArrayWorx biochip reader (Applied Precision).

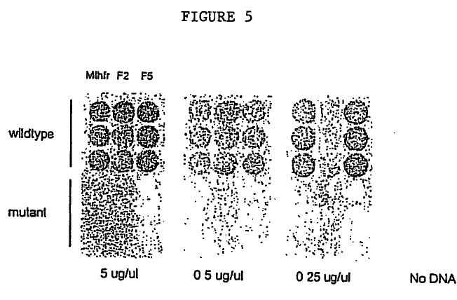

The image shown in FIG. 5 shows that the signals are only present at the capture sites representing the wild-type genotype for all the genes indicating that the samples are wild-type for the three genes (as expected). Moreover, genotyping is possible with as little as 0.25 μg/μL genomic DNA.

### Example 7

### Comparison Study of Low Non-specific Binding of Magnetic Beads Coated with PAA

This example addresses the non-specific binding advantage of the PAA-coated magnetic particles. Amine-modified and carboxylic acid-modified magnetic particles available commercially were compared with the PAA-coated magnetic particles in the context of the Biobarcode assay. Before the assay is run, the level of non-specific binding (background noise) that one may encounter when the magnetic particles come in contact with the co-loaded nanoparticle probes is measured. The experiment in this example shows that the only particles that exhibit non-specific binding similar to the control are the ones coated with PAA and that the commercially available particles are not acceptable. Without being bound by any theory of operation, the surprising reduction in non-specific binding may result from the negatively charged polymer passivating the magnetic bead surface and preventing close contact between the nanoparticle and the magnetic bead. However, the negative charge may not be sufficient as shown by the carboxylic acid-modified beads. The polymeric nature of PAA may also play an important role possibly in sterically shielding close contact.

In the context of the barcode assay, co-loaded probes contain barcodes that are released from the ‘magnetic particle-target-nanoparticle probe’ construct at the end of the target binding assay. The barcodes serve as surrogate targets and because there may be 100-1000 barcodes, or more, associated with each co-loaded probe the biobarcode ‘amplification’ allows detection of extremely low target copies. Central to the performance of the biobarcode amplification assay is the ability to eliminate unbound co-loaded probes. These include those free in solution and those bound to the magnetic beads non-specifically. This ensures that the barcodes released barcodes derive only from target binding events. The PAA-coated magnetic beads ensure that the non-specific binding is minimal and that these are superior to commercially available beads.

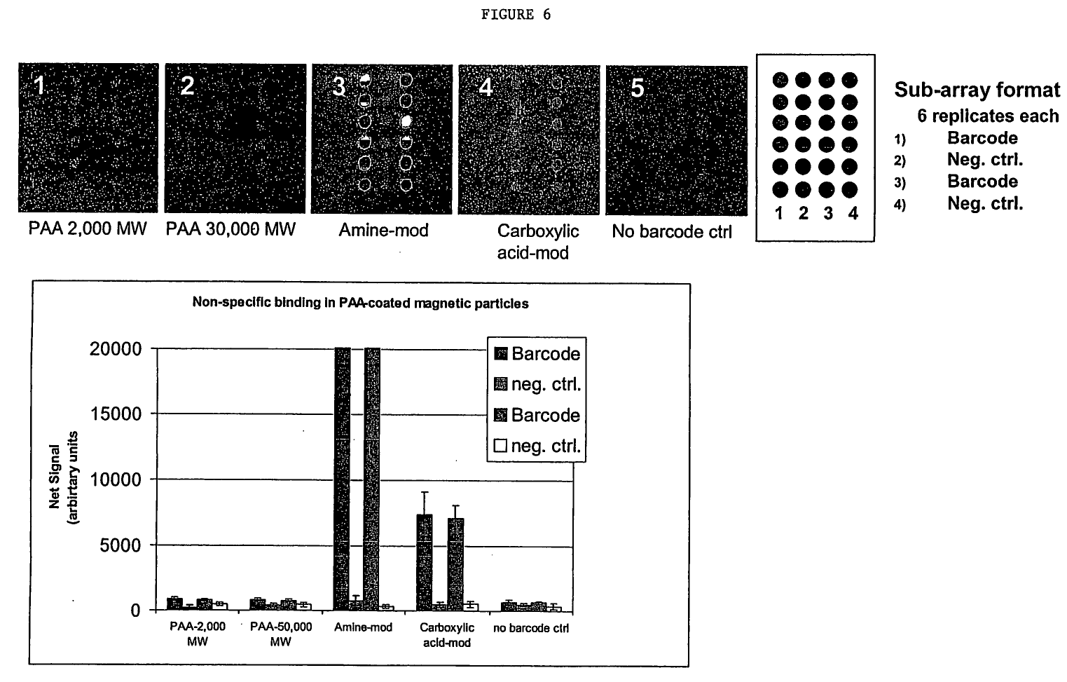

Non-specific binding associated with magnetic beads obtained commercially with amine functional groups and carboxylic acid functional groups were compared with magnetic beads functionalized with PAA. In the experiment, magnetic beads were incubated with a 50 nm diameter co-loaded nanoparticle probe under DNA hybridization conditions. The magnetic beads in each condition were isolated, washed, and heated to release any bound barcodes. The supernatant from each condition was tested in a chip assay designed to detect barcodes at high sensitivity. The chip assay showed nearly saturating signals for the barcodes for both amine-modified and the carboxylic acid-modified magnetic beads indicating that the co-loaded probes associated with the magnetic bead and could not be washed under the barcode assay washes. By contrast, the chip assay showed that virtually no barcodes or co-loaded probes were associated with the PAA-coated beads indicating that the surface modification imparted unique properties optimal for the biobarcode assay. See FIG. 6.

Assay details: To 2 ug of the different magnetic beads, coated with PAA (either 3000 MW or 50,000 MW), amine-modified (Polysciences), and carboxylic acid-modified (Polysciences) was added 20×SSC (final conc. 3.2×SSC), formamide (final 27.5% v/v), and 50 nm co-loaded probe (final conc. 100 pM) in a final volume of 20 uL. The barcode loaded on to the 50 nm T30 probe had the following sequence: 5′-AGTGATTTGAATTTTCAAGCACCCATGGTGGTTACCTCTTCTACTAAAAAAAA AAAAAAAAAAAAAAAAAAAAAA-3′ [SEQ ID NO.:10].

The mix was incubated for 30 min at 40° C. and subsequently the magnetic beads were subjected to extensive washing. Briefly, the beads were isolated by using a magnet and the supernatant was removed. This was followed by 2 washes with 0.3 M NaNO₃; 10% v/v formamide and 3 washes with 0.3 M NaNO₃ and 2 washes with 1 M NaNO₃. After the last wash step, 25 uL ddH₂O was added to the beads and the beads were heated at 95° C. for 1 min to release any non-specifically bound barcodes. The supernatant was tested for the presence of barcodes in a chip assay. The chip assay was conducted by using 20 uL of the supernatant from above and 30 uL of a hyb mix to yield a final concentration of 3.2×SSC (final conc. 3.2×SSC), 27.5% v/v formamide, 0.01% SDS, and 1 nM T20 15 nm probe. The 50 uL mix was added to a test array and incubated for 1 h at 40° C. after which the test array was washed buffer for 3 min (2×) at RT successively in Buffer A (0.5 M NaNO₃; 0.04% Tween; 0.01% SDS) and Buffer B (0.5 M NaNO₃). Finally, the washed slide was stained with 2 mL of silver reagent, an admix of Signal enhancement A and B reagents (Nanosphere, Inc.) for ˜5 min, washed in ddH₂O, and dried. The dried slide was imaged on an ArrayWorx biochip reader (Applied Precision) and the quantitation was performed by using GenePix Software (Molecular Devices).

What is claimed is:

1. A method for modifying a substrate surface, the surface comprising optional amino groups, said method comprising:
providing an activated form of polyacrylic acid; and
contacting a substrate surface with the activated form of polyacrylic acid to form an activated polyacrylic acid coated surface.

2. The method of claim 1, wherein amino groups are present on the surface.

3. The method of claim 1, wherein no amino groups are present on the surface.

4. A method for modifying a substrate surface, the surface comprising optional displaceable functional groups, said method comprising:
contacting the substrate surface with polyacrylic acid to form a polyacrylic acid-containing surface; and
activating the polyacrylic acid-containing surface to form an activated form of polyacrylic acid coating on said surface.

5. The method of claim 4 wherein the displaceable functional group is present.

6. The method of claim 4, wherein the displaceable functional group comprises a tosyl or mesyl group.

7. The method of claim 4 wherein the surface does not include a displaceable functional group.

8. The method of any one of claims 1 or 4, wherein the activated form of polyacrylic acid comprises an N-hydroxysuccinimide ester of polyacrylic acid or N-hydroxysulfosuccinimide ester of polyacrylic acid.

9. The method of any one of claims 1 or 4, further comprising contacting at least a portion of the activated polyacrylic acid coated surface with a molecule so as to immobilize the molecule onto the surface.

10. The method according to claim 9 wherein the molecule comprises DNA, RNA, polypeptide, antibody, antigen, carbohydrate, protein, peptide, amino acid, carbohydrate, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, metabolites of or antibodies to any of the above substances.

11. The method of claim 9 wherein nucleic acids and oligonucleotides comprise genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, natural and synthetic nucleic acids.

12. The method of claim 9 wherein the molecule is a member of a specific binding pair comprising antigen and antibody-specific binding pairs, biotin and avidin binding pairs, carbohydrate and lectin bind pairs, complementary nucleotide sequences, complementary peptide sequences, effector and receptor molecules, enzyme cofactor and enzymes, and enzyme inhibitors and enzymes.

13. The method of claim 9 wherein the substrate has a plurality of different molecules attached thereto in an array to allow for the detection of multiple types of target analytes.

14. The method of claim 9 wherein the substrate comprises magnetic beads, glass slides, silica beads, microplate well, beads, polymer membrane, or optical fiber.

15. A modified substrate produced by any one of claims 1 or 4.

16. The modified substrate of claim 15, wherein the substrate comprises nylon, nitrocelluose, activated agarose, diazotized cellulose, latex particles, plastic, polystyrene, glass and polymer coated surfaces.

17. The modified substrate of claim 15, wherein the substrate comprises slides, membranes, microtiter plates, beads, probes, dipsticks, optical fibers, magnetic beads.

18. The modified substrate of claim 15, wherein the modified substrate is an arrayed plate.

19. The modified substrate of claim 15, wherein the modified substrate is a magnetic bead.

20. In an improved method for detecting at least one target analyte in a sample, the improvement comprising contacting the sample with a substrate having a bound molecule comprising a binding complement specific to the target analyte, the substrate prepared by any one of the methods of claims 1 or 4.

21. A method for detecting for at least one target analyte, the target analyte having at least two binding sites, in a sample, the method comprising steps of:
(a) incubating a capture probe, the sample and a detection probe under conditions effective to allow complex formation between the capture probe, the target analyte, and the detection probe, wherein (i) the capture probe comprising a molecule bound to the magnetic bead, the molecule comprising a first binding complement specific to the target analyte, the magnetic bead having a surface modified by any one of the methods of claims 1 or 4, (ii) the detection probe comprises a gold nanoparticle, a second binding complement to the target analyte bound to the nanoparticle, and reporter moieties bound to the nanoparticle;
(b) separating the complex from any unbound detection probe;
(c) selectively releasing at least a portion of the reporter moieties from the complex; and
(d) analyzing the presence or absence of the reporter moieties, wherein the presence or absence of reporter moieties is indicative of the presence or absence of the target analyte.

22. The method of claim 21, wherein the complex is separated from any unbound detection probe by the application of a magnetic field.

23. The method of claim 21, wherein the nanoparticles include oligonucleotides bound thereto, the reporter moieties comprise oligonucleotides complementary to at least a portion of the oligonucleotides, and the reporter moieties are selectively released from the complex by dehybridization.

24. The method of claim 21, wherein the reporter moieties are directly or indirectly bound to the nanoparticles.


SEQUENCE LISTING (10 sequences)

SEQ ID NO: 1 - DNA, 22 bp, Artificial Sequence
  Factor II wt sequence employed in arrayed oligonucleotides.
ctcagcgagcctcaatgctccc

SEQ ID NO: 2 - DNA, 23 bp, Artificial Sequence
  Factor II mut sequence employed in arrayed oligonucleotides.
ctctcagcaagcctcaatgctcc

SEQ ID NO: 3 - DNA, 22 bp, Artificial Sequence
  Mthfr wt sequence employed in arrayed oligonucleotides.
gatgaaatcggctcccgcagac

SEQ ID NO: 4 - DNA, 22 bp, Artificial Sequence
  Mthfr mut sequence employed in arrayed oligonucleotides.
atgaaatcgactcccgcagaca

SEQ ID NO: 5 - DNA, 24 bp, Artificial Sequence
  Factor V wt sequence employed in arrayed oligonucleotides.
tggacaggcgaggaatacaggtat

SEQ ID NO: 6 - DNA, 26 bp, Artificial Sequence
  Factor V mut sequence employed in arrayed oligonucleotides.
ctggacaggcaaggaatacaggtatt

SEQ ID NO: 7 - DNA, 46 bp, Artificial Sequence
  Factor V sequence of the oligonucleotides tethered to the gold nanoparticles.
ccacagaaaatgatgcccagtgcttaacaagaccatactacagtga

SEQ ID NO: 8 - DNA, 47 bp, Artificial Sequence
  Factor II sequence of the oligonucleotides tethered to the gold nanoparticles.
tcctggaaccaatcccgtgaaagaattatttttgtgtttctaaaact

SEQ ID NO: 9 - DNA, 29 bp, Artificial Sequence
  Mthfr sequence of the oligonucleotides tethered to the gold nanoparticles.
ggaagaatgtgtcagcctcaaagaaaagc

SEQ ID NO: 10 - DNA, 75 bp, Artificial Sequence
  Sequence of the barcode on the T30 probe.
agtgatttgaattttcaagcacccatggtggttacctcttctactaaaaaaaaaaaaaaaaaaaaaaaaaaaaaa